(19) United States
(12) Patent Application Publication
(10) Pub. No.: US 2025/0025299 A1
(43) Pub. Date: Jan. 23, 2025

(54) DEVICES, SYSTEMS, AND METHODS FOR DEPLOYING A TRANSCATHETER AORTIC VALVE IMPLANT WITHIN A SURGICAL AORTIC VALVE IMPLANT

(71) Applicant: Boston Scientific Scimed, Inc., Maple Grove, MN (US)

(72) Inventors: Donal Kyne, Galway (IE); Sean O'Sullivan, Galway (IE)

(73) Assignee: Boston Scientific Scimed, Inc., Maple Grove, MN (US)

(21) Appl. No.: 18/777,797

(22) Filed: Jul. 19, 2024

Related U.S. Application Data
(60) Provisional application No. 63/528,128, filed on Jul. 21, 2023.

(51) Int. Cl. A61F 2/24; A61M 25/00; A61M 25/10

(52) U.S. Cl. CPC A61F 2/2433; A61F 2/2418; A61M 25/10; A61M 25/0041; A61M 2025/1079

(57) ABSTRACT

A pigtail catheter may include an elongate shaft having a curled distal tip, a tubular sheath slidably disposed over the elongate shaft, and a compliant expandable member fixed to the elongate shaft and the tubular sheath. A transcatheter aortic valve replacement (TAVR) system may include the pigtail catheter, a TAVR delivery device comprising an elongate shaft having an implant holding portion proximate a distal end thereof, and a TAVR implant disposed within the holding portion in a collapsed configuration, the TAVR implant comprising an expandable framework including a lower crown disposed at an upstream end and an upper crown disposed downstream of 10 the lower crown. The compliant expandable member is configured to identify a downstream extent of a surgical aortic valve implant disposed within an ascending aorta prior to deploying the TAVR implant.

### CROSS-REFERENCE TO RELATED APPLICATIONS

This application claims the benefit of priority of U.S. Provisional Application No. 63/528,128 filed Jul. 21, 2023, the entire disclosure of which is hereby incorporated by reference.

### TECHNICAL FIELD

The disclosure relates generally to medical devices and more particularly to medical devices pertaining to replacement heart valve implants and procedures related thereto.

### BACKGROUND

A wide variety of intracorporeal medical devices have been developed for medical use including, artificial heart valves for repair or replacement of diseased heart valves. Diseases and/or medical conditions that impact the cardiovascular system are prevalent throughout the world. Some relatively common medical conditions may include or be the result of inefficiency, ineffectiveness, or complete failure of one or more of the valves within the heart. Treatment of defective heart valves poses other challenges in that the treatment often requires the repair or outright replacement of the defective valve. As improvements are made to devices and procedures, replacement heart valve implants are being implanted into younger and lower risk patients who in turn have a longer life expectancy. In some cases, these patients may outlive the useful lifespan of their initial replacement heart valve implant.

In such situations, a second replacement heart valve implant procedure may be required or desired. In some cases, the second replacement heart valve implant, such as a transcatheter aortic valve replacement (TAVR) implant, may be implanted directly within the original/initial replacement heart valve implant, such as a surgical aortic valve (SAV) implant. Properly locating the TAVR implant within and/or relative to the SAV implant may be difficult because different landmarks may be used compared to a TAVR procedure placing a TAVR implant directly into the native heart valve. Identifying those landmarks (e.g., elements of the SAV valve) may be difficult because SAV implants are typically formed with polymeric components which are difficult to image using fluoroscopy (the standard technique in TAVR procedures). There is an ongoing need to provide alternative medical devices and/or systems as well as alternative methods for manufacturing and using medical devices and/or systems.

### SUMMARY

In one example, a pigtail catheter may comprise an elongate shaft having a curled distal tip, a tubular sheath slidably disposed over the elongate shaft, and a compliant expandable member fixed to the elongate shaft and the tubular sheath.

In addition or alternatively to any example described herein, the elongate shaft includes a guidewire lumen extending therethrough.

In addition or alternatively to any example described herein, the compliant expandable member is configured to shift between a collapsed configuration and an expanded configuration.

In addition or alternatively to any example described herein, axial translation of the tubular sheath relative to the elongate shaft is configured to shift the compliant expandable member between the collapsed configuration and the expanded configuration.

In addition or alternatively to any example described herein, the compliant expandable member is formed as a cage having a plurality of interstices.

In addition or alternatively to any example described herein, the compliant expandable member is formed from a radiopaque material.

In addition or alternatively to any example described herein, the cage is formed from one or more interwoven filaments defining the plurality of interstices therebetween.

In addition or alternatively to any example described herein, the cage is monolithically formed from a plurality of interconnected struts defining the plurality of interstices therebetween.

In addition or alternatively to any example described herein, the compliant expandable member is formed as an inflatable balloon.

In addition or alternatively to any example described herein, the inflatable balloon includes a radiopaque material.

In addition or alternatively to any example described herein, the inflatable balloon is configured to be selectively filled with an inflation fluid that is radiopaque.

In addition or alternatively to any example described herein, a transcatheter aortic valve replacement (TAVR) system may comprise a pigtail catheter according to any example described herein; a TAVR delivery device comprising an elongate shaft having an implant holding portion proximate a distal end thereof; and a TAVR implant disposed within the holding portion in a collapsed configuration, the TAVR implant comprising an expandable framework including a lower crown disposed at an upstream end and an upper crown disposed downstream of the lower crown.

In addition or alternatively to any example described herein, a transcatheter aortic valve replacement (TAVR) system may comprise a pigtail catheter comprising an elongate shaft having a curled distal tip, a tubular sheath slidably disposed over the elongate shaft, and a compliant expandable member fixed to the elongate shaft and the tubular sheath; a TAVR delivery device comprising an elongate shaft having an implant holding portion proximate a distal end thereof; and a TAVR implant disposed within the holding portion in a collapsed configuration, the TAVR implant comprising an expandable framework including a lower crown disposed at an upstream end and an upper crown disposed downstream of the lower crown.

In addition or alternatively to any example described herein, the compliant expandable member is configured to identify a downstream extent of a surgical aortic valve implant disposed within an ascending aorta prior to deploying the TAVR implant.

In addition or alternatively to any example described herein, the compliant expandable member is disposable radially outward of at least a portion of the surgical aortic valve implant such that a portion of the compliant expandable member is disposed between the surgical aortic valve implant and a wall of the ascending aorta.

In addition or alternatively to any example described herein, the portion of the compliant expandable member disposed between the surgical aortic valve implant and the wall of the ascending aorta is configured to remain in place between the surgical aortic valve implant and the wall of the ascending aorta as the upper crown is deployed downstream of the downstream extent of the surgical aortic valve implant.

In addition or alternatively to any example described herein, a method of deploying a transcatheter aortic valve replacement (TAVR) implant within a surgical aortic valve implant disposed within a native heart valve and an ascending aorta, comprising: advancing a pigtail catheter intravascularly to a position adjacent the surgical aortic valve implant, the pigtail catheter comprising a compliant expandable member proximate a curled distal tip thereof; positioning the curled distal tip of the pigtail catheter radially outward of the surgical aortic valve implant such that the curled distal tip is disposed between the surgical aortic valve implant and a wall of the ascending aorta; shifting the compliant expandable member from a collapsed configuration toward an expanded configuration with a portion of the compliant expandable member disposed between the surgical aortic valve implant and the wall of the ascending aorta; identifying a downstream extent of the surgical aortic valve implant disposed within the ascending aorta with the compliant expandable member using fluoroscopy; and deploying the TAVR implant within the surgical aortic valve implant at a desired position relative to the downstream extent of the surgical aortic valve implant.

In addition or alternatively to any example described herein, the portion of the compliant expandable member disposed between the surgical aortic valve implant and the wall of the ascending aorta is configured to remain in place between the surgical aortic valve implant and the wall of the ascending aorta as the TAVR implant is deployed within the surgical aortic valve implant.

In addition or alternatively to any example described herein, the method may comprise advancing a TAVR delivery device intravascularly to a location adjacent the surgical aortic valve implant.

In addition or alternatively to any example described herein, the TAVR delivery device comprises an elongate shaft having an implant holding portion proximate a distal end thereof, and the TAVR implant is disposed within the implant holding portion in a collapsed configuration.

In addition or alternatively to any example described herein, in the expanded configuration, a portion of the compliant expandable member that is not disposed between the surgical aortic valve implant and the wall of the ascending aorta bulges radially inwardly toward a center of the ascending aorta at the downstream extent of the surgical aortic valve implant.

In addition or alternatively to any example described herein, the TAVR implant comprises an expandable framework including a lower crown disposed at an upstream end and an upper crown disposed downstream of the lower crown.

In addition or alternatively to any example described herein, deploying the TAVR implant within the surgical aortic valve implant at the desired position includes deploying the upper crown immediately downstream of the downstream extent of the surgical aortic valve implant.

In addition or alternatively to any example described herein, the method may comprise removing the pigtail catheter from the ascending aorta after at least partially deploying the TAVR implant within the surgical aortic valve implant.

The above summary of some embodiments, aspects, and/or examples is not intended to describe each disclosed embodiment or every implementation of the present disclosure. The figures and detailed description which follow more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a partial cutaway view illustrating selected aspects of a transcatheter aortic valve replacement (TAVR) implant positioned within a native valve annulus of a heart;

FIG. 2 is a partial cutaway view illustrating selected aspects of a surgical aortic valve (SAV) implant positioned within a native valve annulus of a heart;

FIG. 3 is a partial cutaway view illustrating selected aspects related to deploying a TAVR implant within an SAV implant; and

FIG. 4 illustrates selected aspects of a pigtail catheter according to the disclosure;

FIGS. 4A-4B illustrate selected aspects of the pigtail catheter of FIG. 4;

FIG. 5 illustrates selected aspects of a pigtail catheter according to the disclosure;

FIGS. 6-8 schematically illustrate selected aspects of a method of using the pigtail catheter of FIGS. 4-5 to deploy a TAVR implant within an SAV implant; and

FIG. 9 is a partial cutaway view illustrating selected aspects of a TAVR implant positioned within an SAV implant within a native valve annulus of a heart.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to one feature may be equally referred to all instances and quantities beyond one of said feature unless clearly stated to the contrary. As such, it will be understood that the following discussion may apply equally to any and/or all components for which there are more than one within the device, etc. unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently-such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Additionally, it should be noted that in any given figure, some features may not be shown, or may be shown schematically, for clarity and/or simplicity. Additional details regarding some components and/or method steps may be illustrated in other figures in greater detail. The devices and/or methods disclosed herein may provide a number of desirable features and benefits as described in more detail below.

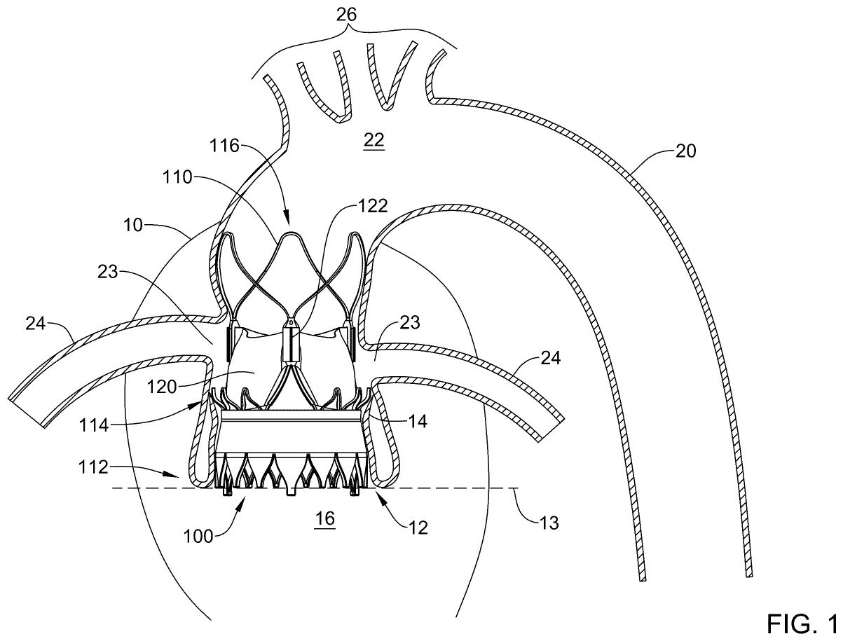

FIG. 1 illustrates a schematic partial cut-away view of a portion of a patient's heart 10 including the aortic valve 12 having native valve leaflets 14 disposed within and/or extending from the native valve annulus, the left ventricle 16, and certain connected vasculature, such as the aorta 20 connected to the aortic valve 12 of the patient's heart 10 by the aortic arch 22 and the ascending aorta, the coronary ostia 23 of the coronary arteries 24, which extend from the aortic sinuses and/or the ascending aorta, and other large arteries 26 (e.g., subclavian and/or carotid arteries, etc.) that extend from the aortic arch 22 to important internal organs. For the purpose of this disclosure, the discussion herein is directed toward treating the aortic valve 12 and will be so described in the interest of brevity. This, however, is not intended to be limiting as the skilled person will recognize that the following discussion may also apply to other heart valves, vessels, and/or treatment locations within a patient.

FIG. 1 further illustrates selected aspects of a transcatheter aortic valve replacement (TAVR) implant 100 positioned within the aortic valve 12 and/or the native valve annulus of the aortic valve 12. In use, the transcatheter aortic valve replacement (TAVR) implant 100 may be implanted (e.g., such as through transcatheter delivery) in the aortic valve 12 of the heart 10. When deploying the transcatheter aortic valve replacement (TAVR) implant 100 within the aortic valve 12 and/or the native valve annulus of the aortic valve 12, such as in a transcatheter aortic valve replacement (TAVR) procedure, the transcatheter aortic valve replacement (TAVR) implant 100 is routinely placed and/or located with respect to an annular plane 13 of the aortic valve 12 and/or the native valve annulus of the aortic valve 12. In some cases, the annular plane 13 may be identified using injected contrast and fluoroscopy.

The transcatheter aortic valve replacement (TAVR) implant 100 may include an expandable framework 110 defining a central lumen. Some suitable but non-limiting examples of materials that may be used to form the expandable framework 110, including but not limited to metals and metal alloys, composites, ceramics, polymers, and the like, are described below. The transcatheter aortic valve replacement (TAVR) implant 100 and/or the expandable framework 110 may be configured to shift between a radially collapsed configuration and a radially expanded configuration. In some embodiments, the expandable framework 110 may be self-expanding. In some embodiments, the expandable framework 110 may be self-biased toward the radially expanded configuration. In some embodiments, the expandable framework 110 may be mechanically expandable. In some embodiments, the expandable framework 110 may be balloon expandable.

In some embodiments, the expandable framework 110 may define a lower crown 112 proximate an upstream end (e.g., and inflow end), an upper crown 114 proximate a downstream end (e.g., an outflow end), and a plurality of stabilization arches 116 extending downstream from the downstream end (e.g., the outflow end). In some embodiments, the lower crown 112 may be disposed at the upstream end (e.g., and inflow end). In some embodiments, the upper crown 114 may be disposed downstream of the lower crown 112. In some embodiments, the upper crown 114 may be disposed at the downstream end (e.g., an outflow end). In some embodiments, the plurality of stabilization arches 116 may extend downstream of and/or away from the upper crown 114 in a direction opposite the lower crown 112. In some embodiments, the upper crown 114 may be disposed longitudinally and/or axially between the lower crown 112 and the plurality of stabilization arches 116.

In some embodiments, the transcatheter aortic valve replacement (TAVR) implant 100 may include a plurality of valve leaflets 120 disposed within the central lumen. The plurality of valve leaflets 120 may be coupled, secured, and/or fixedly attached to the expandable framework 110 at a plurality of commissure posts 122 coupled to and/or integrally formed with the expandable framework 110. In some embodiments, the plurality of commissure posts 122 may be disposed longitudinally and/or axially between the upper crown 114 and the plurality of stabilization arches 116. In some embodiments, the plurality of stabilization arches 116 may extend from the plurality of commissure posts 122.

The transcatheter aortic valve replacement (TAVR) implant 100 can be configured to allow one-way flow through the transcatheter aortic valve replacement (TAVR) implant 100 from an upstream end (e.g., an inflow end) to a downstream end (e.g., an outflow end). The plurality of valve leaflets 120 may be configured to shift between an open position and a closed position. The plurality of valve leaflets 120 may be configured to substantially restrict fluid flow through the transcatheter aortic valve replacement (TAVR) implant 100 in the closed position. The plurality of valve leaflets 120 may move apart from each other in the open position to permit fluid flow through the transcatheter aortic valve replacement (TAVR) implant 100.

In some embodiments, the plurality of valve leaflets 120 may be comprised of a polymer, such as a thermoplastic polymer. In some embodiments, the plurality of valve leaflets 120 may include at least 50 percent by weight of a polymer. In some embodiments, the plurality of valve leaflets 120 may be formed from bovine pericardial or other living tissue. Other configurations and/or materials are also contemplated.

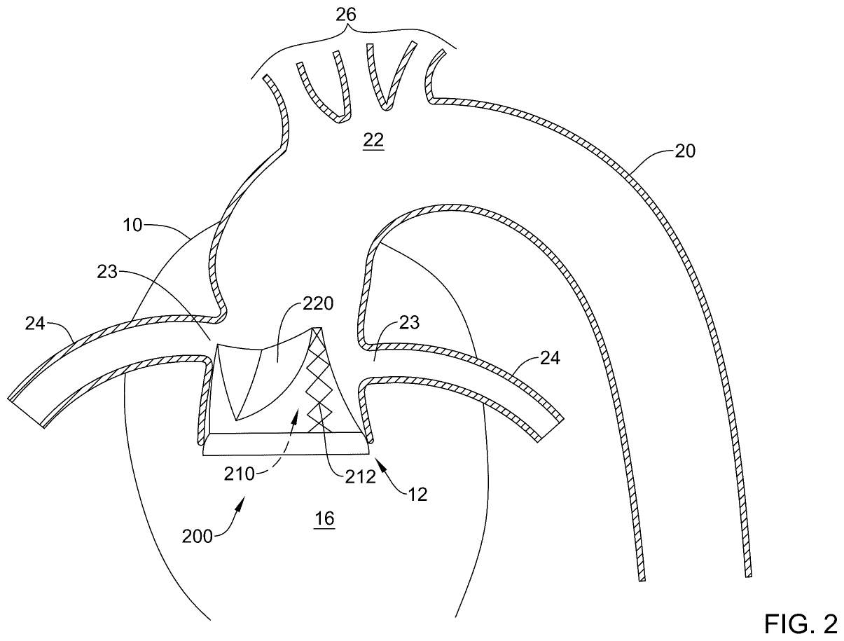

FIG. 2 illustrates selected aspects of a surgical aortic valve (SAV) implant 200 positioned within the aortic valve 12 and/or the native valve annulus of the aortic valve 12. In use, the surgical aortic valve (SAV) implant 200 may be implanted (e.g., such as through surgical delivery) in the aortic valve 12 of the heart 10. In at least some surgical delivery procedures, the native valve leaflets may be excised prior to and/or during implantation of the surgical aortic valve (SAV) implant 200, as shown in FIG. 2. The surgical aortic valve (SAV) implant 200 may be secured to (e.g., sutured to, etc.) the native valve annulus of the aortic valve 12.

In some cases, the surgical aortic valve (SAV) implant 200 may include an expandable framework 210 defining a central lumen. The expandable framework 210 may be commonly formed from polymeric materials. The surgical aortic valve (SAV) implant 200 and/or the expandable framework 210 may be configured to shift between a radially collapsed configuration and a radially expanded configuration. In some embodiments, the expandable framework 210 may be self-expanding. In some embodiments, the expandable framework 210 may be self-biased toward the radially expanded configuration. In some embodiments, the expandable framework 210 may be mechanically expandable. In some embodiments, the expandable framework 210 may be balloon expandable.

In some embodiments, the surgical aortic valve (SAV) implant 200 may include a plurality of valve leaflets 220 coupled, secured, and/or fixedly attached to the expandable framework 210 at a plurality of commissures 212. In some embodiments, the plurality of commissures 212 may include a plurality of commissure posts coupled to and/or integrally formed with the expandable framework 210. The surgical aortic valve (SAV) implant 200 can be configured to allow one-way flow through the surgical aortic valve (SAV) implant 200 from an upstream end (e.g., an inflow end) to a downstream end (e.g., an outflow end). The plurality of valve leaflets 220 may be configured to shift between an open position and a closed position. The plurality of valve leaflets 220 may be configured to substantially restrict fluid flow through the surgical aortic valve (SAV) implant 200 in the closed position. The plurality of valve leaflets 220 may move apart from each other in the open position to permit fluid flow through the surgical aortic valve (SAV) implant 200.

In some embodiments, the plurality of valve leaflets 220 may be comprised of a polymer, such as a thermoplastic polymer. In some embodiments, the plurality of valve leaflets 220 may include at least 50 percent by weight of a polymer. In some embodiments, the plurality of valve leaflets 220 may be formed from bovine pericardial or other living tissue. Other configurations and/or materials are also contemplated.

For the purpose of discussion, this disclosure describes features and/or procedures related to placement of a transcatheter aortic valve replacement (TAVR) implant delivered via a transcatheter procedure (although this is not strictly required) and implanted within a surgical aortic valve (SAV) implant. As illustrated, the figures show the transcatheter aortic valve replacement (TAVR) implant 100 disposed and/or implanted within the surgical aortic valve (SAV) implant 200.

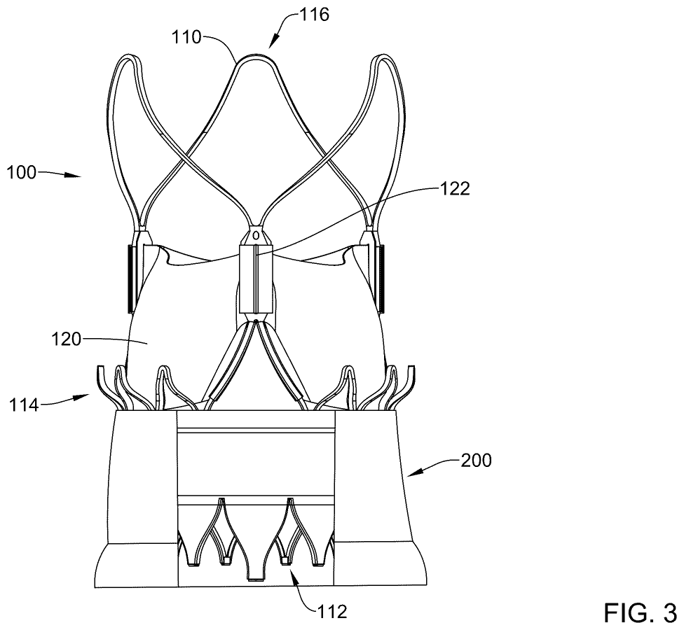

FIG. 3 is a partial cutaway view of the transcatheter aortic valve replacement (TAVR) implant 100 disposed within the surgical aortic valve (SAV) implant 200. As noted above, when positioning the transcatheter aortic valve replacement (TAVR) implant 100 within the aortic valve 12, the annular plane 13 (e.g., FIG. 1) is the landmark that is used. As such, the transcatheter aortic valve replacement (TAVR) implant 100 is placed relative to the annular plane 13. However, when positioning the transcatheter aortic valve replacement (TAVR) implant 100 within the surgical aortic valve (SAV) implant 200, the annular plane 13 may or may not be the most appropriate and/or most accurate landmark. In order to achieve proper function and/or sealing, the transcatheter aortic valve replacement (TAVR) implant 100 should be positioned relative to the surgical aortic valve (SAV) implant 200 instead of relative to the annular plane 13. For example, the surgical aortic valve (SAV) implant 200 may be misaligned with and/or offset from (e.g., positioned upstream or downstream from) the annular plane 13. More particularly, the transcatheter aortic valve replacement (TAVR) implant 100 should be positioned relative to a downstream extent of the surgical aortic valve (SAV) implant 200.

If the transcatheter aortic valve replacement (TAVR) implant 100 is positioned too deep within the surgical aortic valve (SAV) implant 200 and/or too far upstream relative to the surgical aortic valve (SAV) implant 200, the commissure posts of the surgical aortic valve (SAV) implant 200 may prevent the expandable framework 110 of the transcatheter aortic valve replacement (TAVR) implant 100 from completely opening to the expanded configuration and/or interference with the upper crown 114 may prevent proper sealing of the transcatheter aortic valve replacement (TAVR) implant 100 against the surgical aortic valve (SAV) implant 200.

Accordingly, when positioning the transcatheter aortic valve replacement (TAVR) implant 100 within the surgical aortic valve (SAV) implant 200, the upper crown 114 should be positioned immediately downstream of the downstream extent of the surgical aortic valve (SAV) implant 200, as shown in FIG. 3. This will permit the expandable framework 110 of the transcatheter aortic valve replacement (TAVR) implant 100 to deploy to the expanded configuration while making a good seal with the surgical aortic valve (SAV) implant 200.

While FIG. 3 appears to show a close correlation between the lower crown 112 and the upstream end of the surgical aortic valve (SAV) implant 200, this is not necessarily true in every situation. Additionally, the upstream end of the surgical aortic valve (SAV) implant 200 may or may not be aligned with the annular plane 13 (not shown in FIG. 3). As such, the downstream extent of the surgical aortic valve (SAV) implant 200 is the most appropriate landmark to use when positioning the transcatheter aortic valve replacement (TAVR) implant 100 within the surgical aortic valve (SAV) implant 200. In at least some embodiments, the downstream extent of the surgical aortic valve (SAV) implant 200 may be defined by and/or may correlate to the downstream ends of the commissure posts. However, accurately identifying the downstream extent of the surgical aortic valve (SAV) implant 200 and/or the downstream ends of the commissure posts of the surgical aortic valve (SAV) implant 200 may be difficult due to the construction of the surgical aortic valve (SAV) implant 200. The expandable framework and the commissure posts of the surgical aortic valve (SAV) implant 200 are commonly made from a polymeric material that may be difficult to image and/or identify using fluoroscopy.

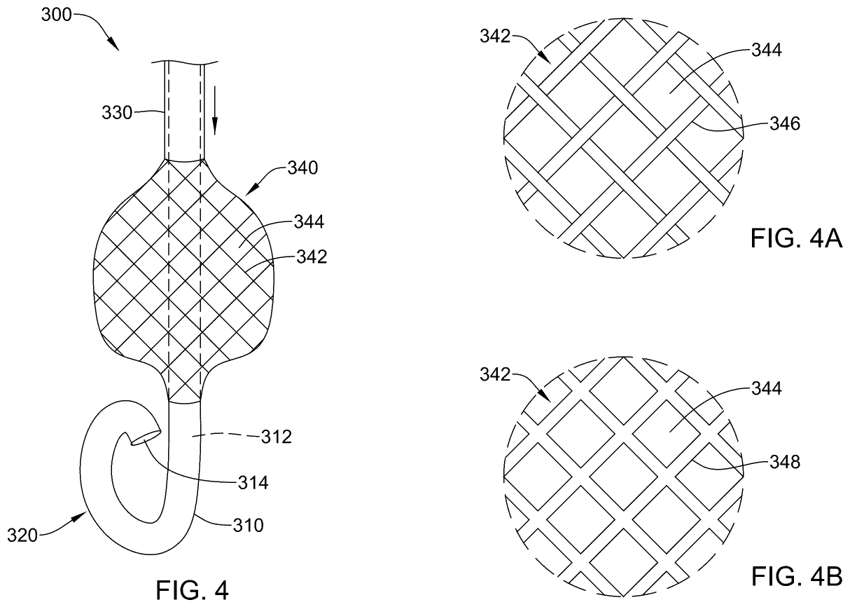

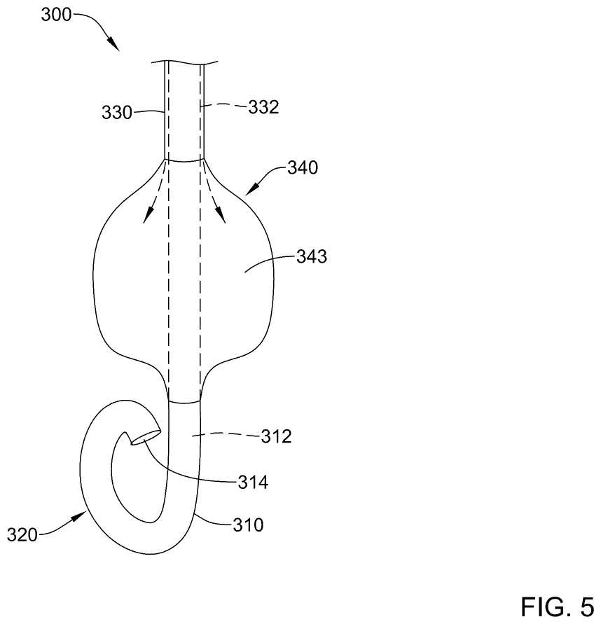

FIGS. 4-5 illustrate selected aspects of a pigtail catheter 300 that may be useful for identifying the downstream extent of the surgical aortic valve (SAV) implant 200 and/or the downstream ends of the commissure posts of the surgical aortic valve (SAV) implant 200. In some embodiments, the pigtail catheter 300 may comprise an elongate shaft 310 having a curled distal tip 320. In some embodiments, the curled distal tip 320 may be configured to assume and/or biased toward a preformed curled shape (e.g., a pigtail shape) in an unconstrained configuration.

The pigtail catheter 300 may comprise a tubular sheath 330 slidably disposed over the elongate shaft 310. The elongate shaft 310 may be slidably disposed within the tubular sheath 330. In some embodiments, the pigtail catheter 300 may comprise a compliant expandable member 340 fixed to the elongate shaft 310 and the tubular sheath 330.

In some embodiments, the elongate shaft 310 may include a guidewire lumen 312 extending therethrough to a distal opening 314. In some embodiments, the guidewire lumen 312 may extend to a proximal end (not shown) of the elongate shaft 310 and/or the pigtail catheter 300. For example, the pigtail catheter 300 may be configured as an over-the-wire (OTW) catheter. In some embodiments, the pigtail catheter 300 may be configured as a single-operator-exchange (SOE) catheter. Other configurations are also contemplated. In some embodiments, the guidewire lumen 312 may be configured to slidably receive a guidewire therein. In some embodiments, the pigtail catheter 300 may be configured to be slidably advanced over a guidewire disposed within the guidewire lumen 312.

In some embodiments, the compliant expandable member 340 may be configured to shift between a collapsed configuration (not shown) and an expanded configuration, shown in FIGS. 4-5. In the collapsed configuration, the compliant expandable member 340 may extend substantially parallel to the elongate shaft 310 and/or the tubular sheath 330. In the collapsed configuration, the compliant expandable member 340 may be axially elongated and/or radially collapsed compared to the expanded configuration. In the expanded configuration, the compliant expandable member 340 may extend radially outward from the elongate shaft 310 and/or the tubular sheath 330. In the expanded configuration, the compliant expandable member 340 may be axially shortened and/or radially expanded compared to the collapsed configuration.

In some embodiments, the compliant expandable member 340 may be disposed proximal of the curled distal tip 320. In some embodiments, the compliant expandable member 340 may be disposed immediately proximal of the curled distal tip 320. In some embodiments, the compliant expandable member 340 may be disposed immediately distal of the tubular sheath 330. In some embodiments, the compliant expandable member 340 may be directly attached to the tubular sheath 330 and/or a distal end of the tubular sheath 330. In some embodiments, the compliant expandable member 340 may be directly attached to the elongate shaft 310. In some embodiments, the compliant expandable member 340 may be directly attached to the elongate shaft 310 proximal of the curled distal tip 320. In some embodiments, the compliant expandable member 340 may be directly attached to the elongate shaft 310 immediately proximal of the curled distal tip 320. In some embodiments, the compliant expandable member 340 may be directly attached to the curled distal tip 320.

In some embodiments, axial translation of the tubular sheath 330 relative to the elongate shaft 310 is configured to shift the compliant expandable member 340 between the collapsed configuration and the expanded configuration. In some embodiments, proximal axial translation of the tubular sheath 330 relative to the elongate shaft 310 may be configured to shift the compliant expandable member 340 toward and/or to the collapsed configuration. In some embodiments, distal axial translation of the tubular sheath 330 relative to the elongate shaft 310 may be configured to shift the compliant expandable member 340 toward and/or to the expanded configuration. Other configurations are also contemplated.

In some embodiments, the compliant expandable member 340 may be monolithically and/or integrally formed with the elongate shaft 310. In some embodiments, the compliant expandable member 340 may be monolithically and/or integrally formed with the tubular sheath 330. In some embodiments, the compliant expandable member 340 may be formed separately from the elongate shaft 310 and the tubular sheath 330 and attached thereto during assembly of the pigtail catheter 300. Other configurations, including combinations thereof, are also contemplated.

In some embodiments, the compliant expandable member 340 may be formed as a cage 342 having a plurality of interstices 344, as shown in FIG. 4. In some embodiments, the compliant expandable member 340 may be formed from a radiopaque material. In some embodiments, the compliant expandable member 340 may be formed from a metallic material. In some embodiments, the compliant expandable member 340 may be formed from a polymeric material doped with a radiopaque element. Other configurations are also contemplated. In some embodiments, the cage 342 may be formed from one or more interwoven filaments 346 defining the plurality of interstices 344 therebetween, as seen in FIG. 4A. In some embodiments, the cage 342 may be monolithically formed from a plurality of interconnected struts 348 defining the plurality of interstices 344 therebetween, as seen in FIG. 4B. Other configurations are also contemplated.

In some embodiments, the compliant expandable member 340 may be formed as an inflatable balloon 343, as shown in FIG. 5. In some embodiments, the inflatable balloon 343 may include a radiopaque material. In some embodiments, the inflatable balloon 343 may be formed from a polymeric material doped with a radiopaque element. In some embodiments, the inflatable balloon 343 may be configured to be selectively filled with an inflation fluid, or an inflation media, which is radiopaque and/or includes a radiopaque element mixed therein. Other configurations are also contemplated. In some embodiments, the tubular sheath 330 may include and/or may define an inflation lumen 332 in fluid communication with the inflatable balloon 343. In some embodiments, the inflation lumen 332 may be formed within a wall of the tubular sheath 330. In some embodiments, the inflation lumen 332 may be formed from an annular space between an inner surface of the tubular sheath 330 and an outer surface of the elongate shaft 310. Other configurations, including multiple inflation lumens, are also contemplated.

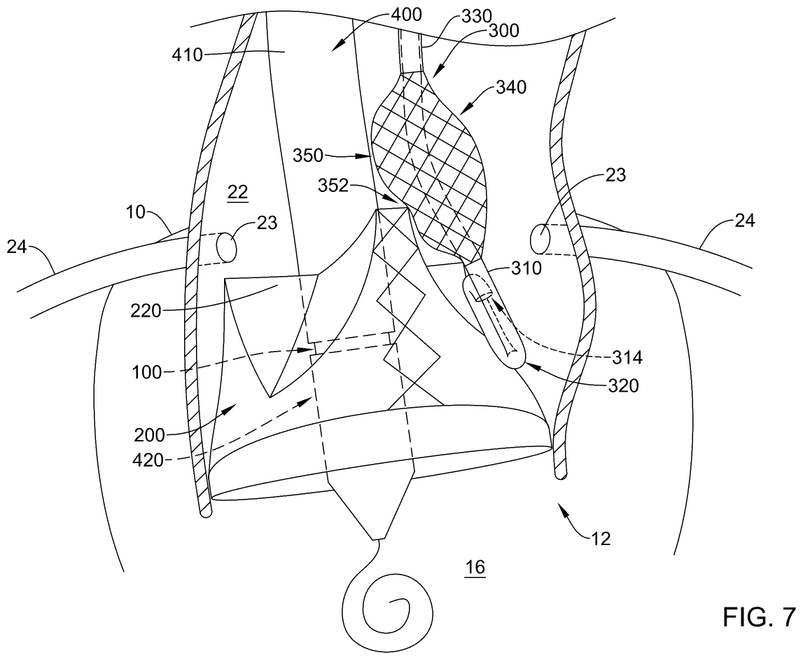

In some embodiments, a transcatheter aortic valve replacement (TAVR) system may comprise the pigtail catheter 300 described herein, a transcatheter aortic valve replacement (TAVR) delivery device 400 (e.g., FIG. 7) comprising an elongate shaft 410 having an implant holding portion 420 proximate a distal end thereof, and the transcatheter aortic valve replacement (TAVR) implant 100 disposed within the implant holding portion 420 in the collapsed configuration. In some embodiments, the elongate shaft 410 may comprise a tubular member. In some embodiments, the elongate shaft 410 may comprise a plurality of tubular members. Other configurations are also contemplated. The transcatheter aortic valve replacement (TAVR) implant 100 may comprise the expandable framework 110 including the lower crown 112 disposed at the upstream end and the upper crown 114 disposed downstream of the lower crown 112, as described herein. The compliant expandable member 340 may be configured to identify the downstream extent of the surgical aortic valve (SAV) disposed within the ascending aorta under fluoroscopy prior to deploying the transcatheter aortic valve replacement (TAVR) implant 100.

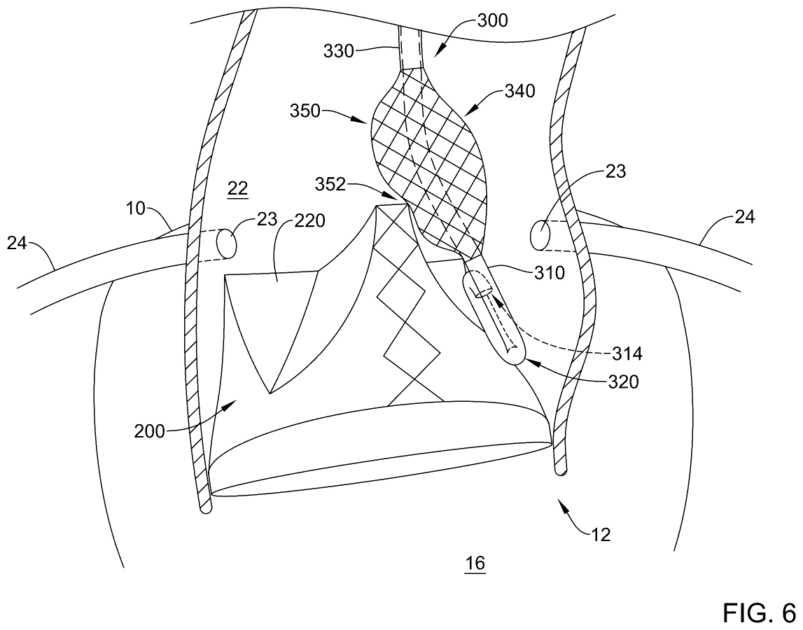

FIGS. 6-9 schematically illustrate selected aspects of a method of deploying the transcatheter aortic valve replacement (TAVR) implant 100 within the surgical aortic valve (SAV) implant 200 within a native heart valve (e.g., the aortic valve 12) and the ascending aorta.

In some embodiments, the method may comprise advancing the pigtail catheter 300 intravascularly to a position adjacent the surgical aortic valve (SAV) implant 200, as shown in FIG. 6. The pigtail catheter 300 may comprise a compliant expandable member 340 proximate a curled distal tip 320 thereof, as described herein. In some embodiments, the method may comprise advancing a guidewire intravascularly to the position adjacent the surgical aortic valve (SAV) implant 200, and subsequently thereafter advancing the pigtail catheter 300 intravascularly over the guidewire to the position adjacent the surgical aortic valve (SAV) implant 200. In some embodiments, the pigtail catheter 300 may be advanced within a delivery sheath to the position adjacent the surgical aortic valve (SAV) implant 200, either alone or over the guidewire, and subsequently deployed from the delivery sheath at the position adjacent the surgical aortic valve (SAV) implant 200. In some embodiments, the curled distal tip 320 may be held or constrained in a straightened configuration during advancement to the position adjacent the surgical aortic valve (SAV) implant 200. In some embodiments, the delivery sheath may hold or constrain the curled distal tip 320 in the straightened configuration, and the delivery sheath may be retracted proximally relative to the pigtail catheter 300, thereby permitting the curled distal tip 320 to assume an unconstrained configuration. Other configurations, including but not limited to the pigtail catheter 300 being formed from a shape memory material configured to shift to the preformed curled shape or the unconstrained configuration when exposed to a stimulus (e.g., temperature, etc.), are also contemplated.

In some embodiments, the method may comprise positioning the curled distal tip 320 of the pigtail catheter 300 radially outward of the surgical aortic valve (SAV) implant 200 such that the curled distal tip 320 is disposed between the surgical aortic valve (SAV) implant 200 and a wall of the ascending aorta, as shown in FIG. 6. In some embodiments, the method may comprise positioning the curled distal tip 320 of the pigtail catheter 300 within the non-coronary cusp. In some embodiments, the method may comprise positioning the curled distal tip 320 of the pigtail catheter 300 at the bottom of the non-coronary cusp. In some embodiments, the compliant expandable member 340 may be disposable radially outward of at least a portion of the surgical aortic valve (SAV) implant 200 such that a portion of the compliant expandable member 340 is disposed between the surgical aortic valve (SAV) implant 200 and the wall of the ascending aorta.

In some embodiments, the method may comprise shifting the compliant expandable member 340 from the collapsed configuration toward the expanded configuration with the portion of the compliant expandable member 340 disposed between the surgical aortic valve (SAV) implant 200 and the wall of the ascending aorta. In some embodiments, shifting the compliant expandable member 340 from the collapsed configuration toward the expanded configuration may cause the compliant expandable member 340 to fill the non-coronary cusp and wrap around the commissure post(s) of the of the surgical aortic valve (SAV) implant 200.

In some embodiments, the method may comprise identifying the downstream extent of the surgical aortic valve (SAV) implant 200 and/or the commissure post(s) of the surgical aortic valve (SAV) implant 200 disposed within the ascending aorta with the compliant expandable member 340 using fluoroscopy. As the compliant expandable member 340 expands over and/or around the top and/or the downstream extent of the surgical aortic valve (SAV) implant 200 and/or the commissure post(s) of the surgical aortic valve (SAV) implant 200, a bulge 350 and/or a ledge 352 may form in the compliant expandable member 340 that is visible via fluoroscopy. In some embodiments, in the expanded configuration, a portion of the compliant expandable member 340 that is not disposed between the surgical aortic valve (SAV) implant 200 and the wall of the ascending aorta may bulge radially inward toward a center of the ascending aorta at the downstream extent of the surgical aortic valve (SAV) implant 200. As such, the bulge 350 and/or the ledge 352 may be used to identify the downstream extent of the surgical aortic valve (SAV) implant 200 and/or the commissure post(s) of the surgical aortic valve (SAV) implant 200.

In some embodiments, the method may comprise advancing the transcatheter aortic valve replacement (TAVR) delivery device 400 intravascularly to a location adjacent the surgical aortic valve (SAV) implant 200, as seen in FIG. 7. As discussed herein, the transcatheter aortic valve replacement (TAVR) delivery device 400 may comprise the elongate shaft 410 having the implant holding portion 420 proximate a distal end thereof. The transcatheter aortic valve replacement (TAVR) implant 100 may be disposed within the implant holding portion 420 of the transcatheter aortic valve replacement (TAVR) delivery device 400 in the collapsed configuration.

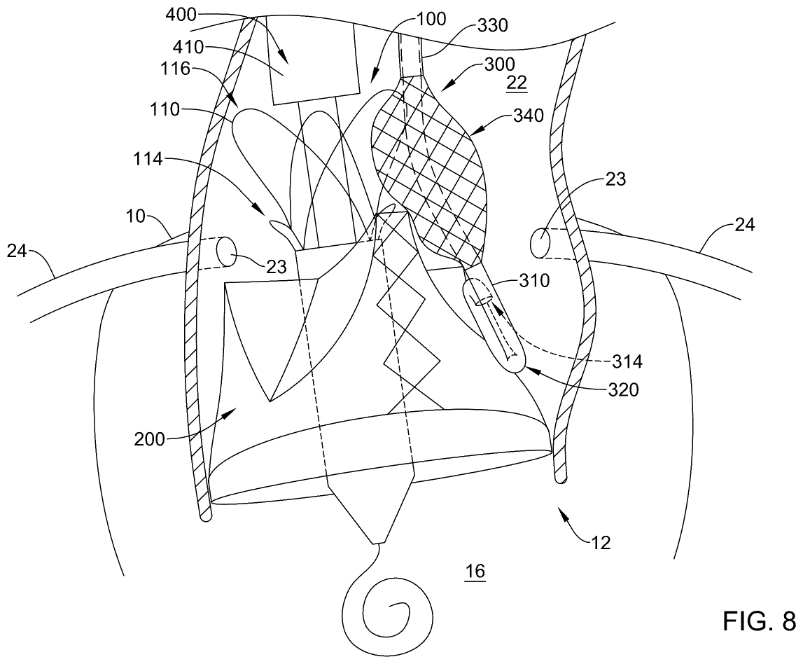

In some embodiments, the method may comprise deploying the transcatheter aortic valve replacement (TAVR) implant 100 within the surgical aortic valve (SAV) implant 200 at a desired position relative to the downstream extent of the surgical aortic valve (SAV) implant 200 and/or the commissure post(s) of the surgical aortic valve (SAV) implant 200. In some embodiments, deploying the transcatheter aortic valve replacement (TAVR) implant 100 within the surgical aortic valve (SAV) implant 200 at the desired position relative to the downstream extent of the surgical aortic valve (SAV) implant 200 and/or the commissure post(s) of the surgical aortic valve (SAV) implant 200 may include deploying the upper crown 114 of the transcatheter aortic valve replacement (TAVR) implant 100 immediately downstream of the downstream extent of the surgical aortic valve (SAV) implant 200 and/or the commissure post(s) of the surgical aortic valve (SAV) implant 200, as seen in FIG. 8. It is noted that in FIG. 8, the transcatheter aortic valve replacement (TAVR) implant 100 is shown partially deployed.

In some embodiments, the portion of the compliant expandable member 340 disposed between the surgical aortic valve (SAV) implant 200 and the wall of the ascending aorta may be configured to remain in place between the surgical aortic valve (SAV) implant 200 and the wall of the ascending aorta as the transcatheter aortic valve replacement (TAVR) implant 100 is deployed within the surgical aortic valve (SAV) implant 200, as seen in FIG. 8. In some embodiments, the portion of the compliant expandable member 340 disposed between the surgical aortic valve (SAV) implant 200 and the wall of the ascending aorta may be configured to remain in place between the surgical aortic valve (SAV) implant 200 and the wall of the ascending aorta as the transcatheter aortic valve replacement (TAVR) implant 100 is at least partially deployed within the surgical aortic valve (SAV) implant 200. In some embodiments, the portion of the compliant expandable member 340 disposed between the surgical aortic valve (SAV) implant 200 and the wall of the ascending aorta may be configured to remain in place between the surgical aortic valve (SAV) implant 200 and the wall of the ascending aorta as the transcatheter aortic valve replacement (TAVR) implant 100 is completely deployed within the surgical aortic valve (SAV) implant 200.

In some embodiments, the portion of the compliant expandable member 340 disposed between the surgical aortic valve (SAV) implant 200 and the wall of the ascending aorta may be configured to remain in place between the surgical aortic valve (SAV) implant 200 and the wall of the ascending aorta as the upper crown 114 of the transcatheter aortic valve replacement (TAVR) implant 100 is deployed downstream of the downstream extent of the surgical aortic valve (SAV) implant 200. In some embodiments, the portion of the compliant expandable member 340 disposed between the surgical aortic valve (SAV) implant 200 and the wall of the ascending aorta may be configured to remain in place between the surgical aortic valve (SAV) implant 200 and the wall of the ascending aorta as the upper crown 114 of the transcatheter aortic valve replacement (TAVR) implant 100 is at least partially deployed downstream of the downstream extent of the surgical aortic valve (SAV) implant 200. In some embodiments, the portion of the compliant expandable member 340 disposed between the surgical aortic valve (SAV) implant 200 and the wall of the ascending aorta may be configured to remain in place between the surgical aortic valve (SAV) implant 200 and the wall of the ascending aorta as the upper crown 114 of the transcatheter aortic valve replacement (TAVR) implant 100 is completely deployed downstream of the downstream extent of the surgical aortic valve (SAV) implant 200.

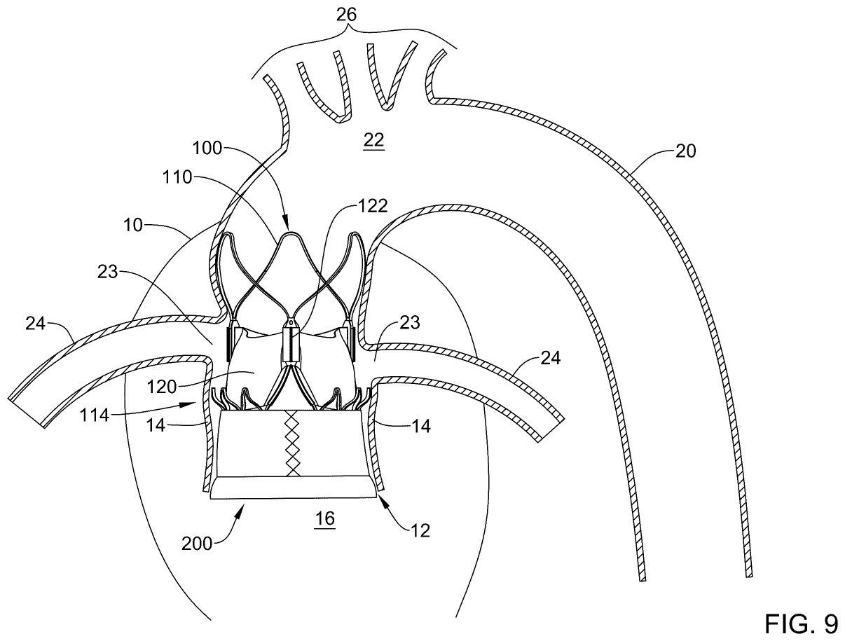

In some embodiments, the method may comprise removing the pigtail catheter 300 and/or the compliant expandable member 340 from the ascending aorta after at least partially deploying the transcatheter aortic valve replacement (TAVR) implant 100 within the surgical aortic valve (SAV) implant 200. In some embodiments, the method may comprise removing the pigtail catheter 300 and/or the compliant expandable member 340 from the ascending aorta after completely deploying the transcatheter aortic valve replacement (TAVR) implant 100 within the surgical aortic valve (SAV) implant 200. FIG. 9 illustrates the transcatheter aortic valve replacement (TAVR) implant 100 deployed within the surgical aortic valve (SAV) implant 200 within the native heart valve (e.g., the aortic valve 12) with the pigtail catheter 300 removed from the ascending aorta.

The materials that can be used for the various components of the system and the various elements thereof disclosed herein may include those commonly associated with medical devices. For simplicity purposes, the following discussion refers to the system. However, this is not intended to limit the devices, components, and methods described herein, as the discussion may be applied to other elements, members, components, or devices disclosed herein, such as, but not limited to, the elongate shaft, the pigtail catheter, the compliant expandable member, the expandable framework, the SAV implant, the TAVR implant, the TAVR delivery device, etc. and/or elements or components thereof.

In some embodiments, the system and/or components thereof may be made from a metal, metal alloy, polymer, a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP). For example, the mixture can contain up to about 6 percent LCP.

Some examples of suitable metals and metal alloys include stainless steel, such as 304 and/or 316 stainless steel and/or variations thereof; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276R, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system and/or components thereof may also be doped with, made of, or otherwise include a radiopaque material. Radiopaque materials are understood to be materials capable of producing a relatively bright image on a fluoroscopy screen or another imaging technique (e.g., ultrasound, etc.) during a medical procedure. This relatively bright image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A pigtail catheter, comprising:
an elongate shaft having a curled distal tip;
a tubular sheath slidably disposed over the elongate shaft; and
a compliant expandable member fixed to the elongate shaft and the tubular sheath.

2. The pigtail catheter of claim 1, wherein the elongate shaft includes a guidewire lumen extending therethrough.

3. The pigtail catheter of claim 1, wherein the compliant expandable member is configured to shift between a collapsed configuration and an expanded configuration.

4. The pigtail catheter of claim 3, wherein axial translation of the tubular sheath relative to the elongate shaft is configured to shift the compliant expandable member between the collapsed configuration and the expanded configuration.

5. The pigtail catheter of claim 1, wherein the compliant expandable member is formed as a cage having a plurality of interstices.

6. The pigtail catheter of claim 5, wherein the compliant expandable member is formed from a radiopaque material.

7. The pigtail catheter of claim 5, wherein the cage is formed from one or more interwoven filaments defining the plurality of interstices therebetween.

8. The pigtail catheter of claim 5, wherein the cage is monolithically formed from a plurality of interconnected struts defining the plurality of interstices therebetween.

9. The pigtail catheter of claim 1, wherein the compliant expandable member is formed as an inflatable balloon.

10. The pigtail catheter of claim 9, wherein the inflatable balloon includes a radiopaque material.

11. The pigtail catheter of claim 9, wherein the inflatable balloon is configured to be selectively filled with an inflation fluid that is radiopaque.

12. A transcatheter aortic valve replacement (TAVR) system, comprising:
a pigtail catheter comprising an elongate shaft having a curled distal tip, a tubular sheath slidably disposed over the elongate shaft, and a compliant expandable member fixed to the elongate shaft and the tubular sheath;
a TAVR delivery device comprising an elongate shaft having an implant holding portion proximate a distal end thereof; and
a TAVR implant disposed within the holding portion in a collapsed configuration, the TAVR implant comprising an expandable framework including a lower crown disposed at an upstream end and an upper crown disposed downstream of the lower crown;
wherein the compliant expandable member is configured to identify a downstream extent of a surgical aortic valve implant disposed within an ascending aorta prior to deploying the TAVR implant.

13. The transcatheter aortic valve replacement system of claim 12, wherein the compliant expandable member is disposable radially outward of at least a portion of the surgical aortic valve implant such that a portion of the compliant expandable member is disposed between the surgical aortic valve implant and a wall of the ascending aorta.

14. The transcatheter aortic valve replacement system of claim 13, wherein the portion of the compliant expandable member disposed between the surgical aortic valve implant and the wall of the ascending aorta is configured to remain in place between the surgical aortic valve implant and the wall of the ascending aorta as the upper crown is deployed downstream of the downstream extent of the surgical aortic valve implant.

15. A method of deploying a transcatheter aortic valve replacement (TAVR) implant within a surgical aortic valve implant disposed within a native heart valve and an ascending aorta, comprising:
advancing a pigtail catheter intravascularly to a position adjacent the surgical aortic valve implant, the pigtail catheter comprising a compliant expandable member proximate a curled distal tip thereof;
positioning the curled distal tip of the pigtail catheter radially outward of the surgical aortic valve implant such that the curled distal tip is disposed between the surgical aortic valve implant and a wall of the ascending aorta;
shifting the compliant expandable member from a collapsed configuration toward an expanded configuration with a portion of the compliant expandable member disposed between the surgical aortic valve implant and the wall of the ascending aorta;
identifying a downstream extent of the surgical aortic valve implant disposed within the ascending aorta with the compliant expandable member using fluoroscopy; and
deploying the TAVR implant within the surgical aortic valve implant at a desired position relative to the downstream extent of the surgical aortic valve implant.

16. The method of claim 15, wherein the portion of the compliant expandable member disposed between the surgical aortic valve implant and the wall of the ascending aorta is configured to remain in place between the surgical aortic valve implant and the wall of the ascending aorta as the TAVR implant is deployed within the surgical aortic valve implant.

17. The method of claim 15, further comprising advancing a TAVR delivery device intravascularly to a location adjacent the surgical aortic valve implant, wherein the TAVR delivery device comprises an elongate shaft having an implant holding portion proximate a distal end thereof, and the TAVR implant is disposed within the implant holding portion in a collapsed configuration.

18. The method of claim 15, wherein in the expanded configuration, a portion of the compliant expandable member that is not disposed between the surgical aortic valve implant and the wall of the ascending aorta bulges radially inwardly toward a center of the ascending aorta at the downstream extent of the surgical aortic valve implant.

19. The method of claim 15, wherein the TAVR implant comprises an expandable framework including a lower crown disposed at an upstream end and an upper crown disposed downstream of the lower crown; and
wherein deploying the TAVR implant within the surgical aortic valve implant at the desired position includes deploying the upper crown immediately downstream of the downstream extent of the surgical aortic valve implant.

20. The method of claim 15, further comprising:
removing the pigtail catheter from the ascending aorta after at least partially deploying the TAVR implant within the surgical aortic valve implant.
